(19) United States
(12) Patent Application Publication
(10) Pub. No.: US 2023/0174793 A1
(43) Pub. Date: Jun. 8, 2023

(54) Self-Disinfecting Frequently Touched Surface

(71) Applicant: Kraton Polymers LLC, Houston, TX (US)

(72) Inventors: Vijay Mhetar, Houston, TX (US); Richard Blackwell, Houston, TX (US); Roger Tocchetto, Houston, TX (US); Sharman McGilbert, Houston, TX (US); Bert Krutzer, Amsterdam (NL)

(73) Assignee: Kraton Polymers LLC, Houston, TX (US)

(21) Appl. No.: 17/995,764

(22) PCT Filed: Apr. 19, 2021
(86) PCT No.: PCT/US2021/070411

§ 371 Date: Oct. 7, 2022

Related U.S. Application Data
(60) Provisional application No. 63/011,576, filed on Apr. 17, 2020.
(60) Provisional application No. 63/019,634, filed on May 4, 2020.
(60) Provisional application No. 63/145,812, filed on Feb. 4, 2021.

(51) Int. Cl. C09D 5/14; A61L 2/232; C08J 7/14; C09D 153/00; C09D 123/34; C09D 181/02

(52) U.S. Cl. CPC C09D 5/14; A61L 2/232; C08J 7/14; C09D 123/34; C09D 153/005; C09D 181/02; C08J 2323/34; C08J 2353/00; C08J 2381/02

(57) ABSTRACT

A frequently-touched surface for use by multiple users includes a surface protected by a laminate structure. The laminate structure includes a self-sterilizing sulfonated polymeric outer layer, sulfonated to a certain % to kill at least 95% microbes within 30 minutes of contact. The self-sterilizing sulfonated polymer layer can be applied to the frequently touched surface by any of coating, lamination, and spraying. The sulfonated polymer in embodiment is selected from perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof. The sulfonated polymer has a degree of sulfonation of at least 10%.

### TECHNICAL FIELD

The disclosure relates to a frequently-touched or used surface, or a “high-touch” surface for use with computer systems for multiple users, having a protective self-disinfecting antimicrobial layer of sulfonated polymer.

### BACKGROUND

We encounter “high-touch” or frequently touched surfaces multiple times in our daily lives. For example, public computer systems or electronic devices for multiple users (not at the same time), such as shared laptops; check-in kiosks at airports; automated teller machines; slot machines at casinos; self-service computers for making reservations for shows, restaurants; self-service stations for hotel guests, car rental customers, or employees to sign-in and sign-out; point-of-sale (POS) monitors in stores and public buildings for information, purchases, etc., all generally include a touch screen display as a user interface. In addition to touch screen displays, locations such as gas stations, public buildings, or even street corners as cross-walk signals, we use touch buttons to enter information and / or for activating a selection. In buildings, touch buttons are used in elevators for floor numbers. All these high-touch surfaces typically require input of commands, with a special stylus or one or more fingers. The high-touch surfaces are exposed to multiple different hands and fingers on a daily basis. This frequent exposure with multiple users leads to transmission of microbes from one user to other user, causing a spread of communicable diseases. The situation may further worsen during a pandemic or during a spread of highly contagious diseases like COVID-19. A person can get sick by touching a surface contaminated with microbes, then use the same fingers to touch their own mouth, nose, or eyes.

There is still a need for a solution to protect high-touch surfaces, to kill and / or prevent accumulation of active microbes on surfaces, preventing the spread of diseases.

### SUMMARY

In a first aspect, a touch panel for a touch screen display is disclosed. The touch panel includes a touch sensitive structure adapted to facilitate a detection of a touching of an area of the touch panel by a user. The touch panel further includes a polymeric layer arranged on the touch sensitive structure. The polymeric layer consists essentially of sulfonated block copolymer, the sulfonated polymer is selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof. The sulfonated polymer has a degree of sulfonation of at least 10%. The sulfonated polymeric protective layer has a thickness of at least 1 µm to kill at least 90% microbes within 120 minutes of contact with the surface.

In some aspects, the sulfonated polymeric layer comprises at least 50 wt.%, more preferably at least 70 wt.%, even more preferably at least 90 wt.%, yet more preferably at least 95 wt.%, still more preferably at least 98 wt.%, even more preferably at least 99 wt.% and most preferably 100 wt.% (i.e. consists) of one or more of the sulfonated polymers.

In embodiments, the touch panel is a capacitive touch panel and the touch sensitive structure includes a transparent conductive layer to facilitate a recognition of a touch by a user. In some embodiments, the touch sensitive structure includes a protective layer deposited on the transparent conductive layer to protect the touch conductive layer from abrasion.

In an embodiment, the touch panel is a resistive touch panel and the touch sensitive structure includes a transparent conductive layer, and a deformable conductive layer arranged above and facing the transparent conductive layer and defines a gap therebetween. The deformable conductive layer compresses in response to a touch of a user or a stylus and contacts the transparent conductive layer indicating a position of the contact.

In embodiments, the touch sensitive structure includes a protective layer deposited on the deformable conductive layer to protect the deformable conductive layer from abrasion. In some embodiments, the polymeric layer is applied onto touch sensitive structure by any of coating, lamination, and spraying. In embodiments, the polymeric layer is formed by coating the touch sensitive structure by one of spray coating and casting. In embodiments, the polymeric layer is removably attached to touch sensitive structure. In some embodiments, the polymeric layer is a peel-off layer adhesively attached to touch sensitive structure.

In an embodiment, the touch panel is a touch panel of a touch screen display of any of a tablet, a monitor, an automated teller machine, and a smartphone.

In yet another aspect, method to prevent transmission of microbes between multiple users of a computer having a touch screen display. The multiple users operates the computer by touching a touch panel to provide input to the computer. The method comprises protecting the surface by applying a self-sterilizing protective sulfonated polymeric layer of a thickness of at least 1 µm to kill at least 90% microbes within 120 minutes of contact with the surface. The sulfonated polymeric layer consists essentially of a sulfonated polymer, the sulfonated polymer being selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof. The sulfonated polymer has a degree of sulfonation of at least 10%.

### DESCRIPTION OF THE DRAWINGS

FIG. 1 is a perspective view of an embodiment of a touch screen display having a touch panel with a protective layer.

FIG. 2 is a front view of an exemplary embodiment of a frequently touched surface, a touch display of a kiosk.

FIG. 3 is a front view of another embodiment of a frequently touched surface, an automated teller machine (ATM) with a touch screen display and key pad(s).

FIG. 4 is a perspective view of yet another embodiment of a frequently touched surface, a handheld game console with touch screen display and a keypad with buttons.

FIG. 5 is front view of an embodiment of a frequently touched surface, a tablet.

FIG. 6 is perspective view of a monitor with a touch screen display.

FIG. 7 is a perspective view of a gaming machine with touch screen displays and touch buttons.

### DETAILED DESCRIPTION

The following terms used the specification have the following meanings:

“Computer” refers an electronic device that accepts input from a user, performs calculations and / or provides information based on the input, e.g., ATM machine, cell phone, tablet, casino machine, etc.

“Effective amount” refers to an amount sufficient to alter, destroy, inactivate, and / or neutralize microbes, e.g., an amount sufficient to sterilize and kill microbes in contact with outer surface of the face panel in a face shield.

“Haze” means the percentage of transmitted light that upon passing through a specimen is scattered greater than 2.5 degrees from the normal. Haze and transmittance can be measured according to ASTM D1003 test method. A higher haze value indicates greater scattering.

“High-touch surfaces” refers to surfaces that are handled frequently throughout the day by numerous people (according definition of the US Centers for Disease Control and Prevention). High-touch surfaces may be referred to highly-touched or frequently-touched surfaces.

“Touch” refers to action or pressure by a finger, a special pen, or a special stylus. The touch can be a light tap, or a swipe by a finger, or a depression or application of pressure by the finger onto the surface.

“Ion Exchange Capacity” or IEC refers to the total active sites or functional groups responsible for ion exchange in a polymer. Generally, a conventional acid-base titration method is used to determine the IEC, see for example International Journal of Hydrogen Energy, Volume 39, Issue 10, Mar. 26, 2014, Pages 5054-5062, “Determination of the ion exchange capacity of anion-selective membrane.” IEC is the inverse of “equivalent weight” or EW, which the weight of the polymer required to provide 1 mole of exchangeable protons.

“Microbes” refers to microorganisms including bacteria, archaea, fungi (yeasts and molds), algae, protozoa, and viruses, with microscopic size.

“Peel-and-stick” or “peel-and-stick film” refers to a laminate having at least two layers, a release layer or liner which can also be a support layer, and another layer containing the sulfonated polymer. The peel-and-stick is self-adhesive, or releasable, or peelable, or removable after being attached to a surface. The release layer is optionally coated with an adhesive which permits it to stick to a surface without glue, paste, or the like, allowing the peel-and-stick to be separable after being applied onto a surface. In embodiments, the layer containing the sulfonated polymer is optionally coated with an adhesive for the layer stick to surface, but is still releasable.

“Laminate structure” can be a structure with a single layer, such as a sulfonated polymeric layer which can adhere to surfaces electrostatically or adhesively, or with at least two layer (e.g., peel-and-stick), with the support side being peeled-off after being applied onto surfaces.

“Die cut” means partial cutting the laminate of a peel-and-stick up to the release liner surface. The cutting can be complete, or it can be partial by perforating. Die cut is typically done with the partial cutting or perforation defining the individual labels or films.

“Releasable” or “separable” bond in the context of layers or surfaces means that the layers or surfaces are generally attached or fastened to each other, yet can be separated with the application of a certain amount of force, and then subsequently refastened or reattached at a later time. In order to be “separable” or “releasable,” the surfaces must be capable of being fastened and separated, and the force applied to separate the layers or surfaces can be applied by hand.

“Surface pH” refers to the pH on the contact surface of the bio-secure material, that results from surface bound moieties e.g., the coating layer. The surface pH can be measured with commercial surface pH measuring instruments, e.g., SenTix™ Sur-electrode from WTW Scientific-Technical Institute GmbH, Weilheim, Germany.

The disclosure relates to a frequent-touched surface having a protective self-sterilizing antimicrobial layer that kills microbes within a predefined duration of contact. The frequent-touched surface employs technology known in the art, e.g., a capacitive sensor, application-specific integrated circuit, controller, digital signal processors, membrane pressure sensitive switches, capacitive switches, etc., as input device for entering letters, numbers, and other symbols into a computer. The computer can be an electronic device, a stand-alone computer, or a networked computer. The frequent-touched surface can be single-touched surface, or multi-touch surface recognizing the presence of more than one point of contact (e.g., one finger) at a time.

The frequent-touched surface is coated with a polymeric layer, or protected with a peel-and-stick polymeric layer comprising a sulfonated polymer. The sulfonated polymer is characterized as self-sterilizing, or having bio-secure properties, particularly suitable for contact with living things, e.g., individuals, animals, that may have infectious and / or contagious diseases, or surfaces or substances that may be exposed to microbes causing infectious and / or contagious diseases.

Self-sterilizing Material - Sulfonated Polymer: As used herein, the term “sulfonated polymer” also covers sulfonate containing polymers, e.g., polystyrene sulfonate.

The sulfonated polymer is selected from the group of perfluorosulfonic acid polymers (e.g., sulfonated tetrafluoroethylene), sulfonated polyolefins, sulfonated polyimides, sulfonated polyamides, sulfonated polyester, polystyrene sulfonates, sulfonated block copolymers, sulfonated polyolefins, sulfonated polysulfones such as polyether sulfone, sulfonated polyketones such as polyether ether ketone, sulfonated polyphenylene ethers, and mixtures thereof.

The sulfonated polymer is characterized as being sufficiently or selectively sulfonated to contain from 10 - 100 mol % sulfonic acid or sulfonate salt functional groups based on the number of monomer units or the block to be sulfonated (“degree of sulfonation”), to kill at least 95% of microbes within 120 minutes of coming into contact with the coating material. In embodiments, the sulfonated polymer has a degree of sulfonation of > 25 mol %, or > 50 mol %, or < 95 mol %, or 25-70 mol %. Degree of sulfonation can be calculated by NMR or ion exchange capacity (IEC).

In embodiments, the sulfonated polymer is a sulfonated tetrafluoroethylene, having a polytetrafluoroethylene (PTFE) backbone; (2) side chains of vinyl ethers (e.g., — O — CF₂ — CF — O — CF₂ — CF₂—) which terminate in sulfonic acid groups in a cluster region.

In embodiments, the sulfonated polymer is a polystyrene sulfonate, examples include potassium polystyrene sulfonate, sodium polystyrene sulfonate, a co-polymer of sodium polystyrene sulfonate and potassium polystyrene sulfonate (e.g., a polystyrene sulfonate copolymer), having a molecular weight of 20,000 to 1,000 ,000 Daltons, or > 25,000 Daltons, or > 40,000 Dalton, or > 50,000, or > 75,000, or > 100,000 Daltons, or > 400,000 Daltons, or < 200,000, or < 800,000 Daltons, or up to 1,500 ,000 Daltons. The polystyrene sulfonate polymers can either be crosslinked or uncrosslinked. In embodiments, the polystyrene sulfonate polymers are uncrosslinked and water soluble.

In embodiments, the sulfonated polymer is a polysulfone, selected from the group of aromatic polysulfones, polyphenylenesulfones, aromatic polyether sulfones, dichlorodiphenoxy sulfones, sulfonated substituted polysulfone polymers, and mixtures thereof. In embodiments, the sulfonated polymer is a sulfonated polyethersulfone copolymer, which can be made with reactants including sulfonate salts such as hydroquinone 2-potassium sulfonate (HPS) with other monomers, e.g., bisphenol A and 4-fluorophenyl sulfone. The degree of sulfonation in the polymer can be controlled with the amount of HPS unit in the polymer backbone.

In embodiments, the sulfonated polymer is a sulfonated polyether ketone. In embodiments, the sulfonated polymer is a sulfonated polyether ketone ketone (SPEKK), obtained by sulfonating a polyether ketone ketone (PEKK). The polyether ketone ketone can be manufactured using diphenyl ether and a benzene dicarbonic acid derivative. The sulfonated PEKK can be available as an alcohol and / or water-soluble product, e.g., for subsequent use to coat the face mask or in spray applications.

In embodiments, the sulfonated polymer is a sulfonated poly(arylene ether) copolymer containing pendant sulfonic acid groups. In embodiments, the sulfonated polymer is a sulfonated poly(2,6-dimethyl-1,4-phenylene oxide), commonly referred to as sulfonated polyphenylene oxide. In embodiments, the sulfonated polymer is a sulfonated poly(4-phenoxybenzoyl-1,4-phenylene) (S-PPBP). In embodiments, the sulfonated polymer is a sulfonated polyphenylene having 2 to 6 pendant sulfonic acid groups per polymer repeat, and characterized as having 0.5 meq (SO₃H)/g of polymer to 5.0 meq (SO₃H)/g polymer, or at least 6 meq/g (SO₃H)/g polymer.

In embodiments, the sulfonated polymer is a sulfonated polyamide, e.g. aliphatic polyamides such nylon-6 and nylon-6,6, partially aromatic polyamides and polyarylamides such as poly(phenyldiamidoterephthalate), provided with sulfonate groups chemically bonded as amine pendant groups to nitrogen atoms in the polymer backbone. The sulfonated polyamide can have a sulfonation level of 20 to up to 100% of the amide group, with the sulfonation throughout the bulk of the polyamide. In embodiments, the sulfonation is limited to a high density of sulfonate groups at the surface, e.g., > 10%, > 20%, > 30%, or > 40%, or up to 100% of the sulfonated amide group at the surface (within 50 nm of the surface).

In embodiments, the sulfonated polymer is a sulfonated polyolefin, containing at least 0.1 meq, or > 2 meq, or > 3 meq, or > 5 meq, or 0.1 to 6 meq of sulfonic acid per gram of polyolefin. In embodiments, the sulfonated polymer is a sulfonated polyethylene. The sulfonated polyolefin can be formed by chlorosulfonation of a solid polyolefin obtained by polymerization of an olefin or a mixture of olefins selected from a group consisting of ethylene, propylene, butene-1,4-methylpentene-1, isobutylene, and styrene. The sulfonyl chloride groups can then be hydrolyzed, for example, in an aqueous base such as potassium hydroxide or in a water dimethylsulfoxide (DMF) mixture to form sulfonic acid groups. In embodiment, the sulfonated polyolefin is formed by submerging or passing polyolefin object in any form of powder, fiber, yarn, woven fabric, a film, a preform, etc., through a liquid containing sulfur trioxide (SO₃), a sulfur trioxide precursor (e.g., chlorosulfonic acid, HSO₃Cl), sulfur dioxide (SO₂), or a mixture thereof. In other embodiments, the polyolefin object is brought into contact with a sulfonating gas, e.g., SO₂ or SO₃, or gaseous reactive precursor, or a sulfonation additive that evolves a gas SOₓ at elevated temperature.

The polyolefin precursor to be sulfonated can be, for example, a poly-α-olefin, such as polyethylene, polypropylene, polybutylene, polyisobutylene, ethylene propylene rubber, or a chlorinated polyolefin (e.g., polyvinylchloride, or PVC), or a polydiene, such as polybutadiene (e.g., poly-1,3-butadiene or poly-1,2-butadiene), polyisoprene, dicyclopentadiene, ethylidene norbornene, or vinyl norbornene, or a homogeneous or heterogeneous composite thereof, or a copolymer thereof (e.g., EPDM rubber, i.e., ethylene propylene diene monomer). In embodiments, the polyolefin is selected from low density polyethylene (LDPE), linear low density polyethylene (LLDPE), very low density polyethylene (VLDPE), high density polyethylene (HDPE), medium density polyethylene (MDPE), high molecular weight polyethylene (HMWPE), and ultra-high molecular weight polyethylene (UHMWPE).

In embodiments, the sulfonated polymer is a sulfonated polyimide, e.g., aromatic polyimides in both thermoplastic and thermosetting forms, having excellent chemical stability and high modulus properties. Sulfonated polyimide can be prepared by condensation polymerization of dianhydrides with diamines, wherein one of the monomeric units contains sulfonic acid, sulfonic acid salt, or sulfonic ester group. The polymer can also be prepared by direct sulfonation of aromatic polyimide precursors, using sulfonation agents such as chlorosulfonic acid, sulfur trioxide and sulfur trioxide complexes. In embodiments, the concentration of sulfonic acid groups in the sulfonated polyimide as measured by ion exchange capacity, IEC, varying from 0.1 meq/g to above 3 meq/g, or at least 6 meq/g.

In embodiments, the sulfonated polymer is a sulfonated polyester, formed by directly sulfonating a polyester resin in any form, e.g., fiber, yarn, woven fabric, film, sheet, and the like, with a sulfuric anhydride-containing gas containing sulfuric anhydride, for a concentration of the sulfone group on the surface of the polyester ranging from 0.1 meq/g to above 3 meq/g, e.g., up to 5 meq/g, or at least 6 meq/g.

In embodiments, the sulfonated polymer is a selectively sulfonated negative-charged anionic block copolymer. The term “selectively sulfonated” definition to include sulfonic acid as well as neutralized sulfonate derivatives. The sulfonate group can be in the form of metal salt, ammonium salt or amine salt.

Depending on the applications and the desired properties, the sulfonated polymer can be modified (or funcationalized). In embodiments, the sulfonated polymer is neutralized with any of various metal counterions, including alkali, alkaline earth, and transition metals, with at least 10% of the sulfonic acid groups being neutralized. In embodiments, the sulfonated polymer is neutralized with inorganic or organic cationic salts, e.g, those based on ammonium, phosphonium, pyridinium, sulfonium and the like. Salts can be monomeric, oligomeric, or polymeric. In embodiments, the sulfonated polymer is neutralized with various primary, secondary, or tertiary amine-containing molecules, with > 10% of the sulfonic acid or sulfonate functional groups being neutralized.

In embodiments, the sulfonic acid or sulfonate functional group is modified by reaction with an effective amount of polyoxyalkyleneamine having molecular weights from 140 to 10,000. Amine-containing neutralizing agents can be mono-functional or multi-functional; monomeric, oligomeric, or polymeric. In alternative embodiments, the sulfonated polymer is modified with alternative anionic functionalities, such as phosphonic acid or acrylic and alkyl acrylic acids.

In embodiments, amine containing polymers are used for the modification of the sulfonated polymers, forming members of a class of materials termed coaservates. In examples, the neutralizing agent is a polymeric amine, e.g., polymers containing benzylamine functionality. Examples include homopolymers and copolymers of 4-dimethylaminostyrene which has been described in U.S. Pat. 9,849,450, incorporated herein by reference. In embodiments, the neutralizing agents are selected from polymers containing vinylbenzylamine functionality, e.g., polymers synthesized from poly-p-methylstyrene containing block copolymers via a bromination-amination strategy, or by direct anionic polymerization of amine containing styrenic monomers. Examples of amine functionalities for functionalization include but are not limited to p - vinylbenzyldimethylamine (BDMA ), p - vinylbenzylpyrrolidine (VBPyr ), p - vinylbenzyl-bis(2-methoxyethyl)amine (VBDEM ), p-vinylbenzylpiperazine (VBMPip ), and p-vinylbenzyldiphenylamine (VBDPA). In embodiments, corresponding phosphorus containing polymers can also be used for the functionalization of the sulfonated polymers.

In embodiments, the monomer or the block containing amine functionality or phosphine functionality can be neutralized with acids or proton donors, creating quaternary ammonium or phosphonium salts. In other embodiments, the sulfonated polymer containing tertiary amine is reacted with alkylhalides to form functional groups, e.g., quaternized salts. In some embodiments, the sulfonated polymer can contain both cationic and anionic functionality to form so-called zwitterionic polymers.

In some embodiments, the sulfonated polymer is a selectively sulfonated negative-charged anionic block copolymer, which “selectively sulfonated” definition to include sulfonic acid as well as neutralized sulfonate derivatives. The sulfonate group can be in the form of metal salt, ammonium salt or amine salt. In embodiments, the sulfonated block polymer has a general configuration A-B-A, (A-B)ₙ(A), (A-B-A)ₙ, (A-B-A)ₙX, (A-B)ₙX, A-D-B, A-B-D, A-D-B-D-A, A-B-D-B-A, (A-D-B)ₙA, (A-B-D)ₙA (A-D-B)ₙX, (A-B-D)ₙX or mixtures thereof; where n is an integer from 0 to 30, or 2 to 20 in embodiments; and X is a coupling agent residue. Each A and D block is a polymer block resistant to sulfonation. Each B block is susceptible to sulfonation. For configurations with multiple A, B or D blocks, the plurality of A blocks, B blocks, or D blocks can be the same or different.

In embodiments, the A blocks are one or more segments selected from polymerized (i) para-substituted styrene monomers, (ii) ethylene, (iii) alpha olefins of 3 to 18 carbon atoms; (iv) 1,3-cyclodiene monomers, (v) monomers of conjugated dienes having a vinyl content less than 35 mol percent prior to hydrogenation, (vi) acrylic esters, (vii) methacrylic esters, and (viii) mixtures thereof. If the A segments are polymers of 1,3-cyclodiene or conjugated dienes, the segments will be hydrogenated subsequent to polymerization of the block copolymer and before sulfonation of the block copolymer. The A blocks may also contain up to 15 mol % of the vinyl aromatic monomers such as those present in the B blocks.

In embodiments, the A block is selected from para-substituted styrene monomers selected from para-methylstyrene, para-ethylstyrene, para-n-propylstyrene, para-iso-propylstyrene, para-n-butylstyrene, para-sec-butylstyrene, para-iso-butylstyrene, para-t-butylstyrene, isomers of para-decylstyrene, isomers of para-dodecylstyrene and mixtures of the above monomers. Examples of para-substituted styrene monomers include para-t-butylstyrene and para-methylstyrene, with para-t-butylstyrene being most preferred. Monomers may be mixtures of monomers, depending on the particular source. In embodiments, the overall purity of the para-substituted styrene monomers be at least 90%-wt., or > 95%-wt., or > 98%-wt. of the para-substituted styrene monomer.

In embodiments, the block B comprises segments of one or more polymerized vinyl aromatic monomers selected from unsubstituted styrene monomer, ortho-substituted styrene monomers, meta-substituted styrene monomers, alpha-methylstyrene monomer, 1,1-diphenylethylene monomer, 1,2-diphenylethylene monomer, and mixtures thereof. In addition to the monomers and polymers noted, in embodiments the B blocks also comprises a hydrogenated copolymer of such monomer (s) with a conjugated diene selected from 1,3-butadiene, isoprene and mixtures thereof, having a vinyl content of between 20 and 80 mol percent. These copolymers with hydrogenated dienes can be any of random copolymers, tapered copolymers, block copolymers or controlled distribution copolymers. The block B is selectively sulfonated, containing from about 10 to about 100 mol % sulfonic acid or sulfonate salt functional groups based on the number of monomer units. In embodiments, the degree of sulfonation in the B block ranges from 10 to 95 mol%, or 15-80 mol%, or 20-70 mol%, or 25-60 mol%, or > 20 mol%, or > 50 mol%.

The D block comprises a hydrogenated polymer or copolymer of a conjugated diene selected from isoprene, 1,3-butadiene and mixtures thereof. In other examples, the D block is any of an acrylate, a silicone polymer, or a polymer of isobutylene with a number average molecular weight of > 1000, or >2000, or >4000, or >6000.

The coupling agent X is selected from coupling agents known in the art, including polyalkenyl coupling agents, dihaloalkanes, silicon halides, siloxanes, multifunctional epoxides, silica compounds, esters of monohydric alcohols with carboxylic acids, (e.g. methylbenzoate and dimethyl adipate) and epoxidized oils.

The antimicrobial and mechanical properties of the sulfonated block copolymer can be varied and controlled by varying the amount of sulfonation, the degree of neutralization of the sulfonic acid groups to the sulfonated salts, as well as controlling the location of the sulfonated group(s) in the polymer. In embodiments and depending on the applications, e.g., one with the need for water dispersity / solubility, or at the other spectrum, one with the need for sufficient durability with constant wiping with water based cleaners, the sulfonated block copolymer can be selectively sulfonated for desired water dispersity properties or mechanical properties, e.g., having the sulfonic acid functional groups attached to the inner blocks or middle blocks, or in the outer blocks of a sulfonated block copolymer, as in U.S. Pat. No. US8084546, incorporated by reference. If the outer (hard) blocks are sulfonated, upon exposure to water, hydration of the hard domains may result in plasticization of those domains and softening, allowing dispersion or solubility.

The sulfonated copolymer in embodiments is as disclosed in Patent Publication Nos. US9861941, US8263713, US8445631, US8012539, US8377514, US8377515, US7737224, US8383735, US7919565, US8003733, US8058353, US7981970, US8329827, US8084546, US8383735, US10202494, and US10228168, the relevant portions are incorporated herein by reference.

In embodiments, the sulfonated block copolymer has a general configuration A-B-(B-A)₁₋₅, wherein each A is a non-elastomeric sulfonated monovinyl arene polymer block and each B is a substantially saturated elastomeric alpha-olefin polymer block, said block copolymer being sulfonated to an extent sufficient to provide at least 1% by weight of sulfur in the total polymer and up to one sulfonated constituent for each monovinyl arene unit. The sulfonated polymer can be used in the form of their acid, alkali metal salt, ammonium salt or amine salt.

In embodiments, the sulfonated block copolymer is a sulfonated polystyrene-polyisoprene-polystyrene, sulfonated in the center segment. In embodiments, the sulfonated block copolymer is a sulfonated t-butylstyrene / isoprene random copolymer with C═C sites in their backbone. In embodiments, the sulfonated polymer is a sulfonated SBR (styrene butadiene rubber) as disclosed in US 6,110,616 incorporated by reference. In embodiments, the sulfonated polymer is a water dispersible BAB triblock, with B being a hydrophobic block such as alkyl or (if it is sulfonated, it becomes hydrophilic) poly(t-butyl styrene) and A being a hydrophilic block such as sulfonated poly(vinyl toluene) as disclosed in US 4,505,827 incorporated by reference. In embodiments, the sulfonated block copolymer is a functionalized, selectively hydrogenated block copolymer having at least one alkenyl arene polymer block A and at least one substantially completely, hydrogenated conjugated diene polymer block B, with substantially all of the sulfonic functional groups grafted to alkenyl arene polymer block A (as disclosed in US 5516831, incorporated by reference). In embodiments, the sulfonated polymer is a water-soluble polymer, a sulfonated diblock polymer of t-butyl styrene / styrene, or a sulfonated triblock polymer of t-butyl styrene -styrene - t-butyl styrene as disclosed in US 4,492,785 incorporated by reference. In embodiments, the sulfonated block copolymer is a partially hydrogenated block copolymer.

In embodiments, the sulfonated polymer is a midblock-sulfonated triblock copolymer, or a midblock-sulfonated pentablock copolymer or, e.g., a poly(p-tert-butylstyrene-b-styrenesulfonate -b-p-tert-butylstyrene), or a poly[tert-butylstyrene-b-(ethylene-alt-propylene)-b-(styrenesulfonate)-b-(ethylene-alt-propylene)-b-tert-butylstyrene.

In embodiments, the sulfonated polymer contains > 15 mol %, or > 25 mol %, or > 30 mol %, or > 40 mol %, or > 60 mol % sulfonic acid or sulfonate salt functional groups based on the number of monomer units in the polymer that are available or susceptible for sulfonation, e.g., the styrene monomers.

In embodiments, the sulfonated polymer has an ion exchange capacity of > 0.5 meq/g, or > 0.75 meq/g, or > 1.0 meq/g, or > 1.5 meq/g, or > 2.0 meq/g, or > 2.5 meq/g, or < 5.0 meq/g.

Optional Additives: In embodiments, the sulfonated polymer further contains or can be complexed with, or otherwise form mixtures, compounds, etc. with, antibiotics such as butylparaben and triclosan, e.g., antimicrobial surfactants, lipids, nanoparticles, peptides, antibiotics or antiviral drugs, tertiary sulfonium, quaternary ammonium and phosphonium containing polymers, chitosan and other naturally occurring antimicrobial polymers, ionexchange resins, metallic-based micro and nano-structured materials such as silver, copper, zinc and titanium and their oxides, for enhanced antimicrobial effectiveness.

In embodiments, the sulfonated polymer further comprises additives for decorative or safety effects, e.g., luminescent additives such as phosphorescent and fluorescence that would help or enable the sulfonated polymer layer to illuminate.

In embodiments, the optional additives are optical brighteners additives that illuminate under a special UV or black light tracer, allowing for physical inspections to verify that intended surfaces are coated or have remained intact, offering the intended antimicrobial / self-disinfecting effects.

In embodiments, the optical additives are UV stabilizers, e.g., UV absorbers, quenchers known in the art.

In embodiments, the sulfonated polymer further comprises additives that would help signal or give an indicator of its antimicrobial effects with a color change pH indicator. Examples include Thymol Blue, Methyl Orange, Bromocresol Green, Methyl Red, Bromothymol Blue, Phenol Red, and Phenol-phthalein. A color change means a change in hue, from a light to a darker color or vice versa. A color indicator may indicate if a recharge, regeneration, or reactivation of the antimicrobial activity of the protective layer is recommended. The color indicator is incorporated in a sufficient amount so that a noticeable change in color hue is observed immediately when there is a change in the effectiveness of the sulfonated polymeric material. For example, when its surface pH is raised above a certain level such as 2.0 (different pathogens have different pH responses), the change is known right away. In embodiments, the amount of color indicator ranges from 0.1 to 20 wt.% of the amount of sulfonated polymer applied as a protective layer on the frequently-touched surface.

In addition to the above optional components, other additives such as plasticizers, tackifiers, surfactants, film forming additives, dyes, pigments, cross-linkers, UV absorbers, catalysts, highly conjugated particles, sheets, or tubes (e.g. carbon black, graphene, carbon nanotubes), etc. may be incorporated in any combination to the extent that they do not reduce the efficacy of the material.

Properties of sulfonated polymer: When applied as a thin protective layer, the sulfonated polymer is characterized as being transparent. Transparency refers to optical clarity, meaning that enough light is transmitted through to allow visualization through the film by an observer. Although some haze or coloration may be presented, such haze or coloration does not substantially interfere with visualization. In embodiments, an antimicrobial sulfonated polymeric layer has a transmission rate of at least 90%, or at least 91%; or clarity of at least 99% or 99.5%, or 85-100%, or 90-100%, or 95-100%; or a haze value of < 1.5%, or < 1.25%, or < 1.0%, or < 0.75%, or 0.5-2%, or 0.5-1.5%. Haze can be measured according to ASTM D-1003. This is in comparison with clear acrylic layers having transmission of 94.5%, haze of 0.1, and clarity of 100%.

In embodiments, the sulfonated polymer is characterized as being sufficiently sulfonated to have an IEC of > 0.5 meq/g, or 1.5-3.5 meq/g, or > 1.25 meq/g, or > 2.2 meq/g, or > 2.5 meq/g, or > 3.0 meq/g, or < 4.0 meq/g.

In embodiments, the sulfonated polymer is characterized as having a surface pH of < 3.0, or < 2.5, or < 2.25, or < 2.0, or < 1.80. It is believed that a sufficiently low surface level, as a result of the presence of sulfonic acid functional groups in the protective layer, would have catastrophic effects on microbes that come in contact with the surface.

In embodiments, the sulfonated polymer works effectively in destroying / inactivating at least 99%, or at least 99.5%, or at least 99.9% of microbes in < 30 minutes of exposure, or < 5 minutes of exposure or contact with microbes, including but not limited to MRSA, vancomycin-resistant Enterococcus faecium, X-MulV, PI-3, SARS-CoV-2, carbapenem-resistant Acinetobacter baumannii, and influenza A virus. In embodiments with polymer containing quaternary ammonium group, the material is effective in killing target microbes including Staphylococcus aureus, Escherichia coli, Staphylococcus albus, Escherichia coli, Rhizoctonia solani, and Fusarium oxysporum. The sulfonated polymer remains effective in killing microbes even after 4 hours, or after 12 hours, or at least 24 hours, or for at least 48 hours.

In embodiments, the sulfonated polymer is a sulfonated block copolymer, e.g., a midblock-sulfonated pentablock copolymer, containing > 40 mol % sulfonic acid or sulfonate salt functional groups based on the number of monomer units. In tests simulating cleaning of the surface of a sulfonated polymeric film, after 2400 cleaning or abrasion cycle, representing 200 days (at least 6 months) in use with 6 cleaning sessions per day (with 4 rubbing motions per session with alcohol and / or quaternary ammonium compounds cleaners).

Methods for Applying Sulfonated Polymeric Protective Layer: The sulfonated polymer can be applied as on the highly-touch surfaces as a coating layer, or as a self-adhesive protective layer or film, having a thickness of < 2000 µm, or > 1 µm, or > 5 µm, or > 10 µm, or < 500 µm, or < 1000 µm, or < 200 µm, or 0.1-2000 µm, or 0.1-500 µm, 1-200 µm. The layer is thin enough to allow activation of the highly touched surface, e.g., a screen or an activating button, yet rigid enough to prevent false activations until the layer is touched.

In embodiments, the sulfonated polymeric layer is applied as a peel-and-stick film. The peel-and-stick film structure in embodiments includes at least one of a transparent conductive layer, an optical film, and an optical compensation film (OCF) layer, which is coated or laminated with the sulfonated polymeric layer. Optical films refer to films that providing desirable transmission and / or reflection properties at least partially though its surface geometry, or arrangements of microlayers of differing refractive index. In embodiments, the peel-and-stick film further comprises a support / release liner. In embodiments, the transparent conductive layer comprises a PEDOT material (poly(3,4-ethylenedioxythiophene)). In embodiments, the transparent conductive layer has a sheet resistance of > 500 ohms / square, or > 1000 ohms / square, or > 3000 ohms / square, or > 6000 ohms / square.

The peel-and-stick film is first peeled off to remove an optional support / release liner (if present), and applied directly onto the highly touched surface. After a period of time, the releasable peel-and-stick with the sulfonated polymeric protective layer can be peeled off the highly touched surface, and replaced with a new peel-and-stick film for protection.

The peel-and-stick structure with a sulfonated polymeric protective layer can be made available as rolls, or large sheets, then cut to size to fit onto the highly-touched surfaces, e.g., touch screens for kiosks, computer monitors, display screens, etc.

In embodiments, the protective sulfonated polymeric layer is applied by first spray coating, or dip coating a scratch resistant film (or laminate) into a solution or dispersion containing the sulfonated polymer, forming a laminate structure with the sulfonated polymer as the outer protective layer. In embodiments, the scratch resistant laminate is capacitive or resistive.

In embodiments of a resistive structure, the laminate comprises at least two layers, with one layer being a resistive material having matrix or lines on the surfaces, with the sheets being separated by air gap. When contact is made to the sulfonated polymeric coating layer on the first resistive sheet, the first sheet is pressed against the 2^{nd} sheet, registering the location of the touch.

In embodiments of a capacitive structure, the laminate comprises two layers with one layer being a transparent conductive layer, separated by a gap in between, with one of the layers being a deformable sheet. The 1^{st} sheet being deformable, when compressed in response to touch (by finger or a stylus pen), contacts the 2^{nd} sheet registering the position of the touch.

In embodiments, the scratch resistant film also functions as an optical film, or an optical compensation film (OCF). The scratch resistant film in embodiments comprises any of polycarbonate, polyester, fluoropolynner, fluorocabon co-polymers, and mixtures thereof.

The laminate structure having a scratch resistant laminate coated with the sulfonated polymer is then cut into size for use with a screen protector frame, e.g., pre-sized into appropriate width and length to cover display screens or surfaces. Attachment mechanisms include Velcro tapes (hook-and-loop assembly), or double-sided adhesive tapes can be applied onto corners or sides of the frame, allowing the screen protector with the sulfonated polymeric layer to be easily installed or removed from the highly touched surface with the lightly adhesive side (releasable side) covering the highly touched buttons.

In embodiments, the peel-and-stick structure is first die-cut into template decal delineating desired design and sized to fit or cover the highly touched surface to be protected, e.g., touch buttons for a keyboard of a kiosk computer, or entry buttons for card payment at a gas pump, etc. To protect surfaces, the peel-and-stick decal is first affixed onto the buttons to be protected with a light touch or pressing of a finger. After the die-cut decal is placed (on the surfaces to be protected), the release liner or backing is subsequently peeled off, leaving the decal with the protective sulfonated polymeric layer behind for protection.

Reference will be made to the figures, showing various embodiments of frequently-touched surfaces with self-disinfecting properties.

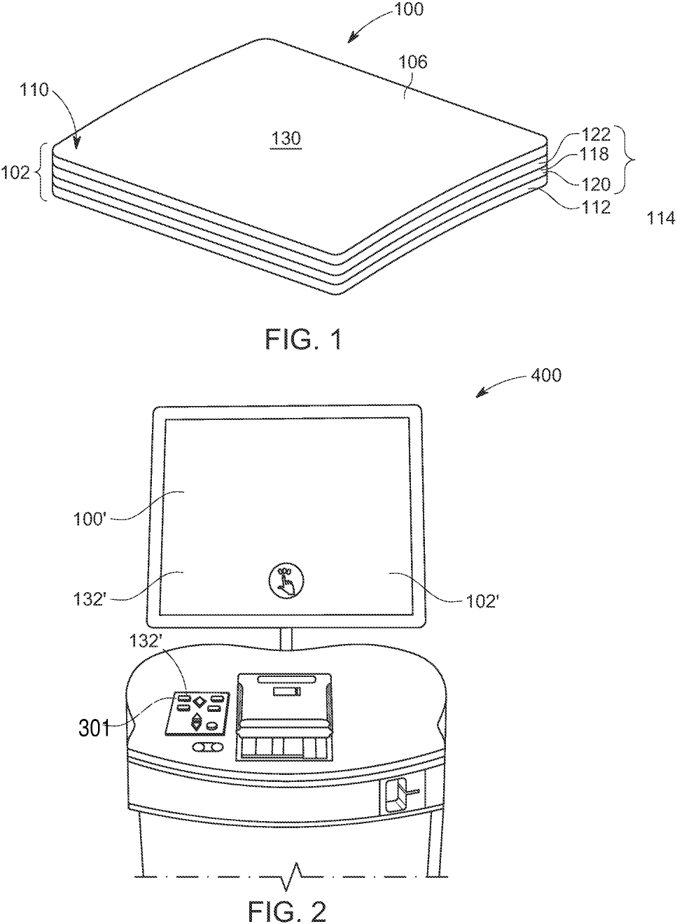

FIG. 1 illustrates a touch screen display 100, with a touch sensitive panel 102 (hereinafter referred to as touch panel 102) with its external surface protected with a sulfonated polymeric layer 106. The touch panel 102 enables the user to interact directly with what is displayed, with input or selection via simple or multi-touch gestures by touching with a special stylus or one or more fingers. The touch panel 102 may be a capacitive touch panel 110, with an insulative substrate 112, such as, glass, plastic or another transparent medium, and a touch sensitive structure 114 the substrate 112. The touch sensitive structure 114 facilitates a detection of a touching of an area of the touch panel 110 by a user, and may include a first layer 118 that is a transparent conductive layer 120 deposited directly on the substrate 112. In embodiments, the first layer 118 may be a tin oxide layer, or silver layer, deposited by methods known in the art, e.g., sputtering, vacuum deposition. The touch sensitive structure 114 includes a conductive pattern, not shown, disposed about a perimeter of the first layer 118 to provide a uniform electric field throughout the first layer 118 in order to establish the point of contact between the display and a finger or stylus. As the human body is also an electrical conductor, touching an external surface of the touch panel 102 results in a distortion of the panel’s/screen’s electrostatic field, measurable as a change in capacitance. In embodiments, the touch sensitive structure 114 may also include a protective layer 122 deposited over the first layer 118 to provide abrasion resistance.

As shown, the touch panel 102 includes the sulfonated polymeric layer 106 protecting the touch sensitive structure 114. However, it is also envisioned that the polymeric layer 106 can be directly laid on the first layer 120. Also in embodiments, the polymeric layer 106 may be a peel-off layer, adhesively attached to touch sensitive structure 114, allowing the polymeric layer 106 to be removed / peeled off after certain usage.

In some embodiments, the polymeric layer 106 may be applied to a transparent substrate, a glass sheet, or a polycarbonate sheet (not shown), which is then removably attached to the touch sensitive structure 114 by an engagement structure, e.g., a hoop and loop fastener assembly (e.g, Velco), snap on tabs, etc.

FIG. 2 illustrates an embodiment of a kiosk 400, which can be for check-in at an airport / a hotel / car rental place, or for inquiries / information at a shopping mall, or a store, a building, etc. The kiosk 400 includes the touch screen display 100′ having a touch sensitive panel 102′ having an antimicrobial protective layer 132′. The protective layer 132′ can be removably attached to the touch sensitive panel 102′ as discussed above. The kiosh 400 has a plurality of touch buttons 301, which can also be protected by the antimicrobial protective layer 132′.

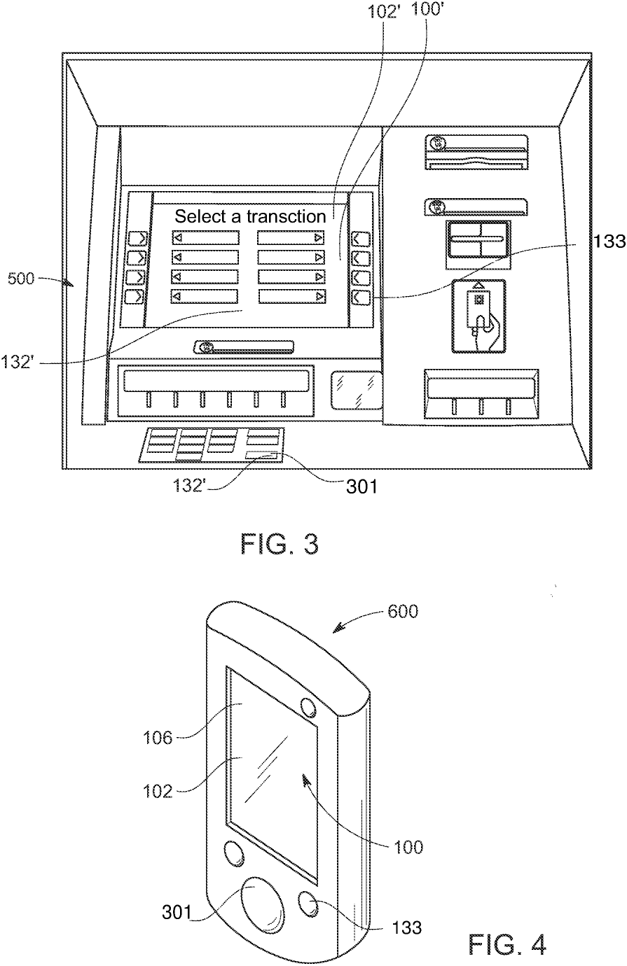

FIG. 3 illustrates an embodiment of an automated teller machine (ATM) 500, with touch screen display 100′ having the touch panel 102′ with the protective layer 132′. As some users may opt to use touch buttons 133 and 301 instead of the touch panel, in embodiments, the touch buttons are also protected by a protective layer 132′.

FIG. 4 refers to a hand-held video game machine 600 with touch screen display 100 having a touch panel 102 with the protective layer 106. In addition to the touch panel, touch buttons 133 and 301 (or the entire front surface of the video game 600 can be protected by the protective layer 132′.

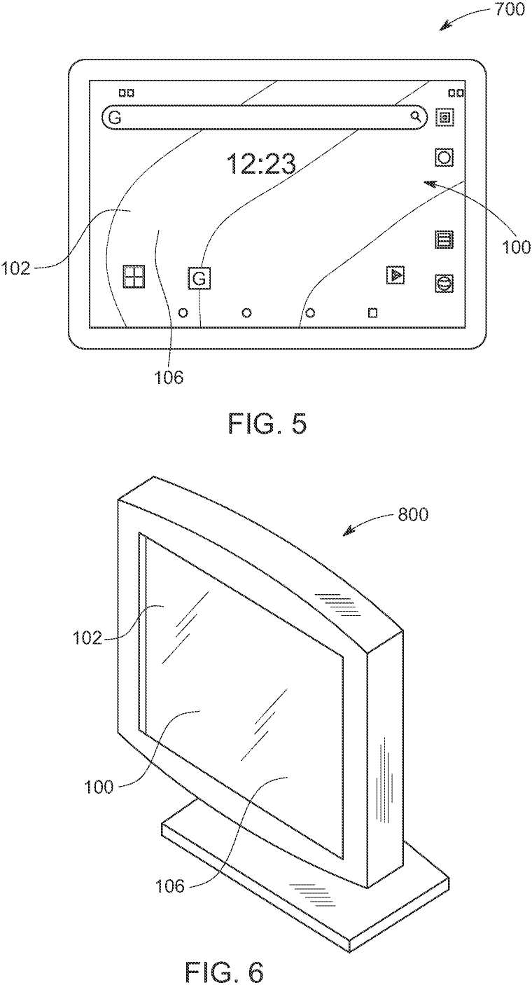

FIG. 5 illustrates a tablet 700 with touch screen display 100, having the touch panel 102 with the protective layer 106.

FIG. 6 illustrates a stand-alone a monitor 800 of a computing unit, with touch screen display 100 having the touch panel 102 with the protective layer 106.

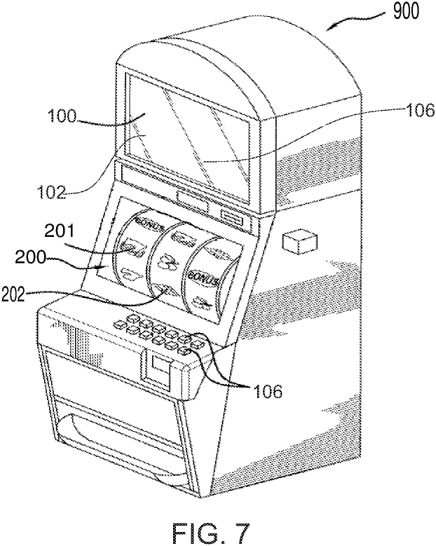

FIG. 7 illustrates an embodiment of a gaming machine 900, e.g., a slot machine, with touch screen display 100′, having a touch panel 102 protected by layer 106. The touch screen may be employed that when touched by a player or a casino operator, performs a function related to the image displayed in the touched area. The player control buttons 133 for functions such as payout (coins), bet, max bet, start (spin reels, etc.) are also protected by the protective layer 106. Reel 200 displays a plurality of symbols as illustrated, e.g., 201 and 202, depending on the selected game, or to show a celebratory display, or to identify the winning symbols. Reel 200 can also be protected with the protective layer 106, allowing the player select a reel or a symbol.

Example 1: Tests were conducted to evaluate antimicrobial efficacy & the long-lasting antiviral properties of sulfonated polymers, film samples of sulfonated penta block copolymer (SPBC) of the structure poly[tert-butylstyrene-b-(ethylene-alt-propylene)-b-(styrene-co-styrene¬sulfonate)-b-(ethylene-alt-propylene)-tert-butylstyrene] with 52% sulfonation were cast out of 1:1 mixture of toluene and 1-propanol. The sulfonated polymer film samples were subjected to abrasion testing of 2200 cycles in the presence of 3 common disinfectants: 1) 70% ethanol, benzalkonium chloride, and quaternary ammonia], and exposure to SARS-CoV-2 virus suspension of concentration 10⁷ pfu/ml.

After 2 hours of contact, viable virus was recovered from each sample by washing twice with 500 µl of DMEM tissue culture media containing 10% serum, and measured by serial dilution plaque assay. Gibco Dulbecco’s Modified Eagle Medium (DMEM) is a basal medium for supporting the growth of many different mammalian cells. The results demonstrate that, after abrasion testing representing approximately one year of cleaning (6 disinfectant wipes/day), surface pro Gibco Dulbecco’s Modified Eagle Medium (DMEM) is a widely used basal medium for supporting the growth of many different mammalian cells.

Example 2. A polyethylene flat sheet of 0.5 mm thick is chlorosulfonated by immersing for six hours at room temperature in a sulfur dioxide/chlorine gas mixture (3:1 volume ratio) in visible light. The chlorosulfonated polyethylene sheet is then immersed in 1N NaOH at 50° C. for two days to hydrolyze the pendant sulfonyl chloride groups (—SO₂Cl) groups to sulfonic groups (—SO₃Na+). The sulfonic acid form is obtained by treating the sheet with 1N HCl at room temperature for four hours. The sheet is then washed with deionized water and dried under vacuum. The milli-equivalence (meq) of sulfonic acid groups per gram of polyethylene is determined by titration with NaOH and found to be 1.69 meq/g. The sulfonated polyethylene sheet can be cut into appropriate sizes for the protection of high-touched surfaces.

Example 3. Dichloromethane (50 mL, 66 gm) and chlorosulfonic acid (between 0.7 and 1.4 gms) are added sequentially to a wide mouth glass bottle (120 mL capacity, 2 inch diameter). 10 mL of this solution are added to dichloromethane (50 mL, 66 gms) in a wide mouth glass jar (410 mL, 3 inch diameter). To this mixture is added a 1 mil (0.001 inch, 0.0025 cm) colorless PPS (Polyphenylene Sulfide) film. The film is allowed to react for various amounts of time at 25° C. while being suspended in the reaction solution. After a variable time of reaction, the black film is then added to distilled water (200 mL) and the film turned light yellow. The film is washed extensively with more water (about 2 liter) and then boiled in water (250 mL) for about 1 hour. The film is then suspended in 1 molar sodium chloride (220 mL) and the amount of sulfonation is determined by titration with 0.01 molar sodium hydroxide to a pH 7 end point. The amount of sulfonation (in meq/g SO₃H) with reaction-time is 0.64 (1 hour), 1.27 (6.5 hours), 1.71 (16 hours), 1.86 (24 hours), 2.31 (48 hours), and 2.6 (60 hours). The sulfonated poly(phenylene sulfide) film can be used for antimicrobial applications as coating materials or as protective films for monitors, keyboards, etc.

As used herein, the term “comprising” means including elements or steps that are identified following that term, but any such elements or steps are not exhaustive, and an embodiment can include other elements or steps. Although the terms “comprising” and “including” have been used herein to describe various aspects, the terms “consisting essentially of” and “consisting of” can be used in place of “comprising” and “including” to provide for more specific aspects of the disclosure and are also disclosed.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained. It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items.

Unless otherwise specified, all technical and scientific terms used herein have the same meanings as commonly understood by one of skill in the art to which the disclosed disclosure belongs. the recitation of a genus of elements, materials or other components, from which an individual component or mixture of components can be selected, is intended to include all possible sub-generic combinations of the listed components and mixtures thereof.

The patentable scope is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims. To an extent not inconsistent herewith, all citations referred to herein are hereby incorporated by reference.

1. A frequently-touched surface for use by multiple users, the frequently-touched surface comprising:
a surface which can be pressed for operation by touching to provide input to a computer; the surface is protected by a sulfonated polymeric layer;
wherein the sulfonated polymeric layer consists essentially of a sulfonated polymer, the sulfonated polymer being selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof;
wherein the sulfonated polymer has a degree of sulfonation of at least 10%;
wherein the sulfonated polymeric protective layer has a thickness of at least 1 µm to kill at least 90% microbes within 120 minutes of contact with the surface.

2. The frequently-touched surface of claim 1, wherein the sulfonated polymer has an ionic exchange capacity (IEC) of > 0.5 meq/g.

3. The frequently-touched surface of claim 1, wherein the sulfonated polymeric layer has a thickness of at least > 5 µm to kill > 95% of microbes within 120 minutes of contact after six months of protection.

4. The frequently-touched surface of claim 1, wherein the sulfonated polymer is selectively sulfonated to contain from 10 - 100 mol % sulfonic acid or sulfonate salt functional groups based on the number of monomer units or blocks in the sulfonated polymer susceptible to sulfonation, for the coating material to kill at least 95% of microbes within 30 minutes of contact.

5. The frequently-touched surface of claim 1, wherein the sulfonated polymer is a selectively sulfonated negative-charged anionic block copolymer having a general configuration of: A-B-A, (A-B)n(A), (A-B-A)n, (A-B-A)ₙX, (A-B)nX, A-D-B, A-B-D, A-D-B-D-A, A-B-D-B-A, (A-D-B)ₙA, (A-B-D)ₙA (A-D-B)ₙX, (A-B-D)ₙX or mixtures thereof, wherein
n is an integer from 0 to 30,
X is a coupling agent residue,
each A and D block is a polymer block resistant to sulfonation,
each B block is susceptible to sulfonation,
the A block is selected from polymerized (i) para-substituted styrene monomers, (ii) ethylene, (iii) alpha olefins of 3 to 18 carbon atoms; (iv) 1,3-cyclodiene monomers, (v) monomers of conjugated dienes having a vinyl content less than 35 mol percent prior to hydrogenation, (vi) acrylic esters, (vii) methacrylic esters, and (viii) mixtures thereof;
the B block is a vinyl aromatic monomer, and
the D block is a hydrogenated polymer or copolymer of a conjugated diene selected from isoprene, 1,3-butadiene and mixtures thereof; and
wherein the block B is selectively sulfonated to contain from 10 - 100 mol % sulfonic acid or sulfonate salt functional groups based on the number of monomer units, for the coating material to kill at least 99% of microbes within 30 minutes of contact.

6. The frequently-touched surface of claim 1, wherein the sulfonated polymeric layer has a surface pH of < 3.0.

7. The frequently-touched surface of claim 1, wherein the sulfonated polymer is neutralized with at least a salt selected from ammonium, phosphonium, pyridinium, and sulfonium salts.

8. The frequently-touched surface of claim 1, wherein the sulfonated polymer is a selectively sulfonated negative-charged anionic block copolymer, having at least one alkenyl arene polymer block A and at least one substantially completely, hydrogenated conjugated diene polymer block B, with substantially all of the sulfonic functional groups grafted to alkenyl arene polymer block A for the block A to be a hydrophilic end-block.

9. The frequently-touched surface of claim 1, wherein the sulfonated polymeric layer has a thickness of 20 to 1000 µm.

10. The frequently-touched surface of any of claims 1-9, wherein the sulfonated polymeric layer is deposited or coated onto a transparent scratch-resistant film to form a laminate structure, prior to application onto the frequently touched surface.

11. The frequently-touched surface of claim 10, wherein the transparent scratch-resistant film is a conductive film or a resistive film.

12. The frequently-touched surface of claims 1-9, wherein the sulfonated polymeric layer is a peel-and-stick film.

13. The frequently-touched surface of claims 1-9, wherein the sulfonated polymeric layer is applied to the frequently-touched surface by any of coating, lamination, and spraying.

14. The frequently-touched surface of claims 1-9, wherein the frequently-touched surface is a touch panel of a touch screen display of any of a tablet, a monitor, an automated teller machine, and a smartphone.

15. A method to prevent transmission of microbes between multiple users of a computer having a touch screen display, wherein the multiple users operates the computer by touching a touch panel to provide input to the computer, the method comprises protecting the surface by applying a self-sterilizing protective sulfonated polymeric layer of a thickness of at least 1 µm to kill at least 90% microbes within 120 minutes of contact with the surface;
wherein the sulfonated polymeric layer consists essentially of a sulfonated polymer, the sulfonated polymer being selected from the group of perfluorosulfonic acid polymers, polystyrene sulfonates, sulfonated block copolymers, sulfonated polyolefins, sulfonated polyimides, sulfonated polyamides, sulfonated polyesters, sulfonated polysulfones, sulfonated polyketones, sulfonated poly(arylene ether), and mixtures thereof;
wherein the sulfonated polymer has a degree of sulfonation of at least 10%.
